(19) United States
(12) Patent Application Publication
(10) Pub. No.: US 2025/0263777 A1
(43) Pub. Date: Aug. 21, 2025

(54) METHODS FOR SEQUENCING LONG POLYNUCLEOTIDES

(71) Applicant: Illumina, Inc., San Diego, CA (US)

(72) Inventors: Aaron Statham, Beaconsfield (AU); Aaron Earl Darling, Hornsby (AU); Kay Jutamat Anantanawat, Parramatta (AU); Kevin Ying, Jannali (AU); Leigh Monahan, Stanmore (AU); Ian Charles, London (GB)

(73) Assignee: Illumina, Inc., San Diego, CA (US)

(21) Appl. No.: 19/202,329

(22) Filed: May 8, 2025

Related U.S. Application Data
(63) Continuation of application No. PCT/US2023/036573, filed on Nov. 1, 2023.
(60) Provisional application No. 63/383,066, filed on Nov. 9, 2022.

(51) Int. Cl. C12Q 1/6806; C12Q 1/6876

(52) U.S. Cl. CPC C12Q 1/6806; C12Q 1/6876

(57) ABSTRACT

A method of determining at least a partial order of fragments derived from a same target template nucleic acid molecule is provided, the method including: a) providing a sample including a target template nucleic acid molecule; b) creating tagged fragments of the target template nucleic acid molecule using sets of tag nucleic acid molecules; c) sequencing at least a portion of the tagged fragments, wherein said portion includes a tag nucleic acid sequence; d) identifying sequences of the tagged fragments that include two or more of the tag nucleic acid sequences that are same, and identifying sequences of the tagged fragments that include two or more of the tag nucleic acid sequences that are different; and e) identifying sequences of the tagged fragments to determine the partial order of the tagged fragments with the target template nucleic acid molecule.

### RELATED APPLICATIONS

The present application is a continuation of PCT/US2023/036573 filed on Nov. 1, 2023, which claims priority to U.S. Provisional Application No. 63/383,066, filed Nov. 9, 2022. The contents of both applications are incorporated herein by reference in their entirety.

### BACKGROUND

### Field

The disclosed technology relates to methods for determining the nucleotide sequence of polynucleotides. More specifically, the disclosed technology relates to methods of tagging long polynucleotides with barcode sequences in an ordered process to allow long reads of template molecules.

### Description

A multitude of technologies are currently available for DNA sequencing, each with different strengths and weaknesses that must be traded off when selecting the most suitable approach for a particular application. For example, so-called second generation sequencing technologies are able to generate highly accurate data at very high throughput and low cost, but can only produce short sequence reads (150 to 600 bp). This limits the ability of these technologies to assemble genomes, since genomic DNA frequently contains repetitive sequences in which individual repeat units are longer than the read length itself. Similarly, short-read data is limited in its capacity to detect structural variants and resolve haplotypes. In addition, some regions can be inherently difficult to sequence at the chemistry level due to issues with secondary structure or high GC content. These have been referred to as “dark” regions (Ebbert et al., 2019).

To address some of the problems with short-read technologies, one strategy has been the development of linked-read methods in which long DNA molecules are barcoded to generate a sparse collection of short fragments for sequencing. This preserves some long-range information, which can help resolve genome assembly breaks, call structural variants and phase haplotypes. However, the short length of individual sequence reads ultimately limits the effectiveness of these approaches. For example, when a series of identical or non-identical repeat elements exist within the span of a linked read molecule, it may not be possible to unambiguously assign individual short reads to different copies of the repeat, with the net effect that variant calling in such repeat regions may remain difficult or error-prone from linked reads.

In contrast to short-read and linked-read approaches, long-read technologies are able to produce continuous sequences that range from several kilobases to several megabases in length. These include sequencing platforms from Pacific Biosciences (PacBio) and Oxford Nanopore Technologies (ONT), which are capable of generating long reads directly from native DNA, as well as “synthetic” long-read approaches in which true end-to-end DNA molecules are reconstructed from short reads. Infinity Long Reads, developed by Longas Technologies, is an example of a synthetic approach that generates continuous long reads. Importantly, both native and synthetic long read technologies are able to routinely produce reads long enough to traverse the most common repetitive elements found in nature, enabling much more contiguous genome assemblies than are currently possible with short-read or linked-read methods.

In practice, read lengths obtained using long-read technologies tend to be limited by the various chemical and physical processes involved in preparing a DNA library and generating the sequence itself. Even the ONT platform, which is capable of sequencing individual DNA molecules up to the megabase scale, typically produces read N50 values on the order of 10-60 kbp (Logsdon et al., 2020). A tradeoff between read length and accuracy is also apparent, with the most highly accurate long read types being limited to around 10-20 kbp. These include PacBio HiFi reads (Wenger et al., 2019) and Infinity Long Reads. Critically, these read lengths are well below the physical length of native DNA molecules (hundreds to thousands of kbp) that can be obtained through careful extraction and purification from many biological sample types.

### SUMMARY

The methods disclosed herein each have several aspects, no single one of which is solely responsible for their desirable attributes. Without limiting the scope of the claims, some prominent features will now be discussed briefly. Numerous other embodiments are also contemplated, including embodiments that have fewer, additional, and/or different components, steps, features, objects, benefits, and advantages. The components, aspects, and steps may also be arranged and ordered differently. After considering this discussion, and particularly after reading the section entitled “Detailed Description”, one will understand how the features of the devices and methods disclosed herein provide advantages over other known devices and methods.

In some embodiments, a method of determining spatial locality of fragments derived from a target template nucleic acid molecule is provided, the method including: a) providing a sample including a target template nucleic acid molecule; b) creating tagged fragments of the target template nucleic acid molecule using sets of tag nucleic acid molecules, wherein: i) each of the sets of tag nucleic acid molecules includes a tag portion, the tag portion including a tag nucleic acid sequence that is unique to that set; ii) the target template nucleic acid molecule is contacted with two or more different sets of the tag nucleic acid molecules; and iii) the tag nucleic acid molecules of each of the sets are spatially associated; wherein each of the tagged fragments include the tag nucleic acid sequence; c) sequencing at least a portion of the tagged fragments, wherein said portion includes the tag nucleic acid sequence; d) identifying sequences of the tagged fragments that include two or more of the tag nucleic acid sequences that are the same; and e) determining the identified tagged fragments as originating from a same spatial locality of the target template nucleic acid molecule.

In some embodiments, a method of determining at least a partial order of fragments derived from a same target template nucleic acid molecule is provided, the method including: a) providing a sample including a target template nucleic acid molecule; b) creating tagged fragments of the target template nucleic acid molecule using sets of tag nucleic acid molecules, wherein: i) each of the sets of tag nucleic acid molecules includes a tag portion including a tag nucleic acid sequence that is substantially unique to that set; ii) the target template nucleic acid molecule is contacted with two or more different sets of tag nucleic acid molecules; and iii) the tag nucleic acid molecules of each of the sets are spatially associated; wherein each end of the tagged fragments includes the tag nucleic acid sequence; c) sequencing at least a portion of the tagged fragments, wherein said portion includes the tag nucleic acid sequence; d) identifying sequences of the tagged fragments that include two or more of the tag nucleic acid sequences that are same, and identifying sequences of the tagged fragments that include two or more of the tag nucleic acid sequences that are different; and e) identifying sequences of the tagged fragments to determine the partial order of the tagged fragments with the target template nucleic acid molecule, wherein: a same first tag nucleic acid sequence at each end likely originated from a same first region of the target template nucleic acid molecule, a same second tag nucleic acid sequence at each end likely originated from a same second region of the target template nucleic acid molecule, and sequences of the tagged fragments which include the first tag nucleic acid sequence at one end and the second tag nucleic acid molecule at the other end originated from a region of the target template nucleic acid molecule intermediate to the first and second regions.

In some embodiments, a method of identifying fragments derived from a same target template nucleic acid molecule is provided, the method including: a) providing a sample including two or more target template nucleic acid molecules; b) creating tagged fragments of the two or more target template nucleic acid molecules using sets of tag nucleic acid molecules, wherein: i) each of the sets of tag nucleic acid molecules includes a tag portion including a tag nucleic acid sequence that is unique to that set; ii) a modal number of different sets of tag nucleic acid molecules which contact each target template nucleic acid molecule is two or more, wherein each set of tag nucleic acid molecules includes a different tag nucleic acid sequence; and iii) a modal number of target template nucleic acid molecules which each set of tag nucleic acid molecules contacts is one, wherein each tagged fragment includes one or more tag nucleic acid sequences; c) sequencing at least a portion of the tagged fragments, wherein said portion includes the tag nucleic acid sequence; and d) identifying sequences of the tagged fragments which have at least one tag nucleic acid sequence in common. In some embodiments, the tagged fragments include a tag nucleic acid sequence at each end.

In some embodiments, step b) includes an amplification step, wherein the tag nucleic acid molecules are primers and include a target binding site capable of hybridising to at least one internal region of a target template nucleic acid molecule, and a tag portion, wherein the tag portion is 5′ to the target binding site. In some embodiments, the amplification step includes PCR amplification. In some embodiments, the amplification step includes isothermal amplification. In some embodiments, the amplification step includes multiple displacement amplification (MDA).

In some embodiments, at least one set of the tag nucleic acid molecules includes tag nucleic acid molecules having two or more different target binding sites. In some embodiments, the target binding sites include degenerate sequences. In some embodiments, the tag nucleic acid molecules are localised in a droplet. In some embodiments, two or more different sets of the tag nucleic acid molecules are located in each droplet.

In some embodiments, the tagged fragments include the tag nucleic acid sequence at each end, and wherein the step d) includes: linking 1) any sequences of the tagged fragments which include a same tag nucleic acid sequence at each end with 2) any sequences of the tagged fragments which include a different tag nucleic acid sequence at each end, wherein one of the different tag nucleic acid sequences is common with the same tag nucleic acid sequences; and determining the order of the tagged fragments within the target template nucleic acid molecule. In some embodiments, the sample further includes one or more additional target template nucleic acid molecules, wherein the step d) further includes identifying sequences of the tagged fragments generated from the step c), the sequences of the tagged fragments including at least one of the tag nucleic acid sequence in common, and wherein the step e) further includes grouping the sequences of the tagged fragments that include at least one of the tag nucleic acid sequences in common to determine the spatial locality of the tagged fragments derived from the same target template nucleic acid molecule.

In some embodiments, the creating of the tagged fragments includes tagmentation. In some embodiments, the tag nucleic acid molecules are immobilised on a solid support.

In some embodiments, the sequencing step includes ligating the ends of the tagged fragments and sequencing the tag nucleic acid sequence in a region of the ligation junction, optionally wherein the sequencing includes sequencing at least 20, at least 30, at least 40, at least 50, at least 60, at least 70, at least 80, at least 90, or at least 100 nucleotides 5′ and/or 3′ of the ligation junction.

In some embodiments, each of the tag nucleic acid molecules includes a common adapter sequence 5′ to the tag portion, wherein each of the tagged fragments includes adapter sequences at 3′ and 5′ ends of the tagged fragment. In some embodiments, the adapter sequences can anneal to one another. In some embodiments, the method further includes a step of amplifying the tagged fragments using primers that are complementary to a portion of the adapter sequence at the 3′ end of each of the tagged fragments. In some embodiments, each of the tag nucleic acid molecules includes an adapter sequence 5′ to the tag portion and an adapter sequence 3′ to the tag portion, wherein the adapter sequence 5′ to the tag portion and the adapter sequence 3′ to the tag portion are the same sequence, and wherein each of the tagged fragments further includes, from each of the 5′ and 3′ ends thereof, a 5′ adapter sequence, the tag nucleic acid sequence, and a 3′ adapter sequence.

In some embodiments, after creating the tagged fragments, the method further includes: extending the 3′ end of any of the tagged fragments, for which the adapter sequence at the 3′ end of the tagged fragment has annealed to the adapter sequence at the 5′ end of the tagged fragment, using a 5′ tag nucleic acid sequence as an extension template to form a concatemeric sequence including the 5′ and a 3′ tag nucleic acid sequences at the 3′ end of the tagged fragment; and sequencing the concatemeric sequence. In some embodiments, the sequencing includes paired-end sequencing. In some embodiments, the sequencing further includes a step of bridge PCR. In some embodiments, the sequencing includes long read sequencing. In some embodiments, the sequencing includes nanopore sequencing. In some embodiments, the sequencing includes circular consensus sequencing. In some embodiments, the sequencing includes synthetic long read sequencing. In some embodiments, the method further includes determining the sequence of the at least one target nucleic acid molecule.

In some embodiments, a method for determining a sequence of at least one target nucleic acid molecule is provided, the method including: a) providing a sample including at least one target nucleic acid molecule; b) creating tagged fragments of the target template nucleic acid molecule using a set of tag nucleic acid molecules, wherein the tag nucleic acid molecules include a single adapter sequence, and wherein each of the fragments includes adapter sequences at 3′ and 5′ ends of the fragments; c) amplifying the fragments generated in step b) using primers that are complementary to a portion of adapter sequence at the 3′ end of each of the fragments; and d) sequencing at least regions of the amplified fragments of the target nucleic acid molecule generated in step c) wherein the tag nucleic acid molecules are conjugated to a solid support, wherein the fragments are created by tagmentation, and wherein the adapter sequences at the 3′ and '5 ends of the fragments can anneal to one another.

In some embodiments, a method for determining a sequence of at least one target nucleic acid molecule is provided, the method including: a) providing a sample including at least one target nucleic acid molecule; b) creating tagged fragments of the at least one target nucleic acid molecule using sets of tag nucleic acid molecules, wherein: i) each of the sets of tag nucleic acid molecules includes: a tag portion including a tag nucleic acid sequence that is unique to that set; and a single adapter sequence, wherein the tag nucleic acid molecules are conjugated to a solid support; ii) the target template nucleic acid molecule is contacted with one or more different sets of tag nucleic acid molecules; and iii) the tag nucleic acid molecules of each of the sets are spatially associated; wherein the fragments are created by tagmentation, wherein each of the fragments includes adapter sequences at 3′ and 5′ ends of the fragments that can anneal to one another, and wherein each of the fragments derived from a same at least one target nucleic acid molecule include a same barcode sequence; c) amplifying the fragments using primers that are complementary to a portion of the adapter sequence at the 3′ end of each fragment; and d) sequencing at least regions of the amplified fragments of the target nucleic acid molecule generated in step c) to provide sequences of the fragments; and e) linking the sequences of the fragments which include the same barcode sequence, thereby to determine the sequence of the at least one target nucleic acid molecule. In some embodiments, the sequencing includes paired-end sequencing. In some embodiments, the sequencing includes a step of bridge PCR. In some embodiments, the sequencing includes long read sequencing.

In some embodiments, a method for determining a sequence of at least one target nucleic acid molecule is provided, the method including: a) providing a sample including at least one target nucleic acid molecule; b) creating tagged fragments of the at least one target nucleic acid molecule using sets of tag nucleic acid molecules, wherein: i) each of the sets of tag nucleic acid molecules includes a tag portion that includes: a tag nucleic acid sequence that is unique to that set; and a single adapter sequence; ii) the target template nucleic acid molecules is contacted with one or more different sets of localised tag nucleic acid molecules; and iii) the tag nucleic acid molecules of each of the sets are spatially associated; wherein each of the fragments includes adapter sequences at 3′ and 5′ ends of the fragments that can anneal to one another, and wherein each of the fragments derived from a same at least one target nucleic acid molecule includes the same barcode sequence; c) amplifying the fragments generated in step ii) using primers that are complementary to a portion of the adapter sequence at the 3′ end of each fragment; and d) sequencing at least regions of the amplified fragments of the target nucleic acid molecule generated in step c) by long-read sequencing to provide long read sequences of the fragments; and e) linking the long read sequences of the fragments to determine the sequence of the at least one target nucleic acid molecule, wherein the tag nucleic acid molecules are conjugated to a solid support, wherein the fragments are created by tagmentation, and wherein the long read sequences of the fragments include a same barcode sequence. In some embodiments, the sequencing includes nanopore sequencing. In some embodiments, the sequencing includes circular consensus sequencing. In some embodiments, the sequencing includes synthetic long read sequencing.

In some embodiments, a method for fragmenting at least one target nucleic acid molecule is provided, the method including: a) providing a sample including at least one target nucleic acid molecule; b) creating tagged fragments of the target template nucleic acid molecule using a set of tag nucleic acid molecules, wherein the tag nucleic acid molecules include a single adapter sequence, wherein each of the fragments includes adapter sequences at 3′ and 5′ ends of the fragments which can anneal to one another; c) amplifying the fragments generated in step b) using primers that are complementary to a portion of adapter sequence at the 3′ end of each fragment; and d) collecting the amplified fragments generated in step c), wherein the tag nucleic acid molecules are conjugated to a solid support, and wherein the fragments are created by tagmentation.

In some embodiments, the method further includes sequencing the fragments. In some embodiments, the tag nucleic acid molecules further include an adapter sequence 5′ to the tag portion. In some embodiments, after the creating of the tagged fragments step, the fragments are amplified using primers capable of hybridising to the adapter sequences. In some embodiments, any of the target template nucleic acid molecule are longer than 10 kb, longer than 20 kb, longer than 30 kb, longer than 40 kb, longer than 50 kb, longer than 60 kb, longer than 70 kb, longer than 80 kb, longer than 90 kb, longer than 100 kb, longer than 200 kb, longer than 300 kb, longer than 400 kb, longer than 500 kb, longer than 750 kb, longer than 1 Mb, longer than 2 Mb, longer than 3 Mb, longer than 4 Mb, longer than 5 Mb, longer than 10 Mb, longer than 20 Mb, longer than 50 Mb, or longer than 100 Mb in length. In some embodiments, the sample includes at least 2, at least 3, at least 4, at least 5, at least 10, at least 20, at least 50, at least 100, at least 200, at least 500, at least 1000, at least 2000, at least 5000, at least 10,000 at least 20,000 at least 50,000 or at least 100,000 target template nucleic acid molecules.

In some embodiments, the method further includes mapping sequences of the fragments to a reference genome. In some embodiments, the method includes creating an assembly graph from the sequences of the fragments.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIGS. 1A and 1B are schematic representations of a long template molecule being tagged with multiple tags according to some non-limiting embodiments of the disclosure.

FIGS. 2A and 2B are schematic representations showing polymerase extension products and partial order information from reads of the long template molecule of FIG. 1A.

FIG. 3 is a flow diagram of a method for determining the spatial locality of fragments derived from a target template nucleic acid molecule according to some non-limiting embodiments of the disclosure.

FIG. 4 is a flow diagram of a method for determining at least a partial order of fragments derived from a target template nucleic acid molecule according to some non- limiting embodiments of the disclosure.

FIG. 5 is a flow diagram of a method for identifying fragments derived from the same target template nucleic acid molecule according to some non-limiting embodiments of the disclosure.

FIG. 6 is a flow diagram of a method for determining a sequence of at least one target nucleic acid molecule according to some non-limiting embodiments of the disclosure.

FIG. 7 is a schematic representation which illustrates the generation of tag pair concatemer sequences via template self-priming according to some non-limiting embodiments of the disclosure.

FIGS. 8A and 8B are line graphs showing the relationship between amplification bias and fragment length from multiple displacement amplification (MDA) according to some non-limiting embodiments of the disclosure with FIG. 8A showing MDA reaction products and the relationship between the abundance and length of the reaction products and FIG. 8B showing the reduction of MDA bias via size selection.

FIG. 9 is a line graph which illustrates the size distribution of fragments generated by barcoded bead tagmentation and subsequent large fragment amplification according to some embodiments.

FIG. 10 is a bar graph which shows an estimated size distribution of bead-barcoded long fragments, based on distances between simple endwall pairs.

FIG. 11 is a diagram which shows long reads that hop between beads in some embodiments.

FIG. 12 is a line graph which shows the size distribution of on-bead M DA products.

### DETAILED DESCRIPTION

All patents, applications, published applications and other publications referred to herein are incorporated herein by reference to the referenced material and in their entireties. If a term or phrase is used herein in a way that is contrary to or otherwise inconsistent with a definition set forth in the patents, applications, published applications and other publications that are herein incorporated by reference, the use herein prevails over the definition that is incorporated herein by reference.

As disclosed herein, embodiments of the invention combine elements of long-read and linked-read approaches to determine the sequences of “long” polynucleotides with additional linkage information, and also ordering information among the linked reads. Using high molecular weight input DNA, a molecular barcoding method, as disclosed herein, is applied to generate a collection of dual-barcoded fragments that contain copies of (potentially overlapping) segments of each much longer starting molecule. These dual-barcoded fragments (typically up to 20 kbp, for example) are then processed for sequencing, either by long read sequencing or via another sequencing process that optionally reads some portion of the fragment and one or both of the associated barcodes. This dual-barcoding process can enable a single long molecule to become barcoded with multiple different barcode sequences, and the collection of barcodes associated with a single long molecule can be ascertained by analysing sequence reads that contain pairs of barcodes. Finally, the barcoding information is used to link the (long) reads together and to provide a partial or total ordering over the reads, thereby enabling the sequence of the original long template molecule to be partially or completely reconstructed.

The methods disclosed herein can provide several important benefits for downstream analysis. In some embodiments, the method may help to resolve large segmental duplications (e.g., >20 kbp) to produce more highly contiguous genome assemblies than comparative short read data. In some embodiments, the methods provide long-range haplotyping information and enable resolution of haplotypes across regions that are inherently difficult to sequence. With respect to long range haplotype phasing, embodiments provide access to a tunable continuum between long-read and linked-read sequencing. In some embodiments, the method is robust against the loss of individual sequence fragments containing barcode pairs because the method creates multiple tagged fragments that associate the same pair of barcodes.

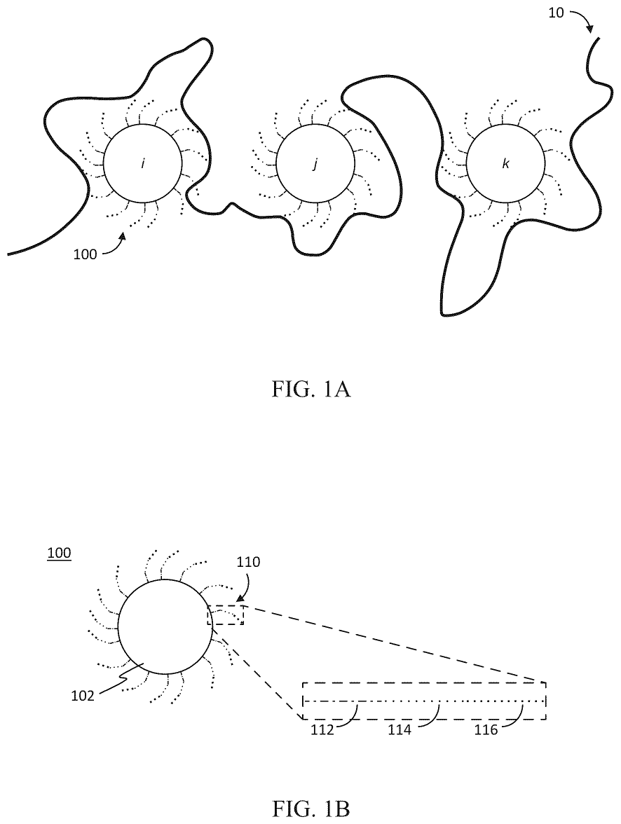

FIG. 1A shows a diagram of barcoding a long template fragment 10 of DNA (>1 kbp, >10 kbp, >50 kbp, etc.) under conditions where portions of the single long template molecule 10 become tagged with two or more short barcode sequences attached to beads i, j and k, with each bead comprising a unique identifier for the template molecule 10, the unique identifier identifying each bead. In some embodiments, the unique identifier includes the barcode sequence. The barcoding reaction may be carried out in conditions that favor a small number of unique identifiers becoming randomly associated with each single polynucleotide molecule. Because of the beads and the respective unique identifiers that identify beads i, j, and k, the order in which the template molecule 10 would bind to the primers attached to each bead is known. In some embodiments, a substrate includes the bead which provides spatial association among one or more of the barcodes on the surface of each bead. The beads themselves diffuse slowly through the solution, so that individual beads (and the barcodes attached thereon) tend to interact with a local part of the template molecule 10 as shown in FIG. 1A. In some embodiments, the template molecule 10 is contacted by a plurality of beads as shown. In some embodiments, the template molecule 10 includes a polynucleotide. In some embodiments, the reaction products are generated in a manner that enables the association between the multiple unique identifiers of the beads and a single template nucleic acid molecule to be determined.

In some embodiments, tag nucleic molecules 110 shown in FIG. 1B (also referred to as unique identifiers and oligo structures interchangeably) are tethered to the solid support 100, which is a physical structure that includes, but is not limited to, a bead, microbead, nanoparticle, or local region of a surface of a substrate. As shown, the tag nucleic molecule 110 includes a tether region 112 a sequencing adapter and barcode 114 (also referred to as a tag interchangeably), and a random primer or adapter primer 116 In some embodiments, the tether region 112 comprises nucleic acids that are designed to couple to the solid support 100 as shown in FIG. 1B. The tag nucleic molecules 110 tethered to the solid support 100 enables the order in which the unique identifiers 110 interact with the template 10 to be determined in terms of the order of the linear nucleic acid template's sequence.

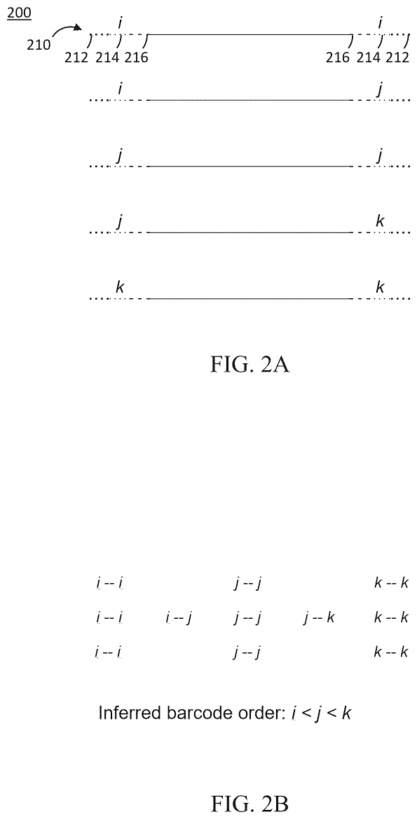

In some embodiments, the long template molecule 10 is tagged with multiple tag nucleic molecules 110 In some embodiments, the long template molecule 10 is tagged with multiple tag nucleic molecules 110, wherein the multiple tag nucleic molecules 110 are tethered to a solid support 100 as shown in FIG. 1B. In some embodiments, the long template molecules 10 (e.g., ≥100 kb in length) are contacted with the solid support 100, wherein the support 100 includes multiple barcoded beads as shown. In some embodiments, each bead 100 includes a set of identical tag nucleic molecules 110 (each including one barcode out of a pool of typically many millions) that become attached to the sequence of the template molecule 10 either via primer extension or via tagmentation. In some embodiments, each bead 100 is designated and/or identified by their respective barcodes 114 as shown. For example, if the barcodes 114 of the beads 100 include barcodes i, j, and k as shown in FIG. 1A, then the resulting reaction products 200 will be of the form of polynucleotides having barcode sequences i - - - i, i - - - j, j - - - j, j - - - k, k - - - k as shown in FIG. 2A.

In some embodiments, the reaction products 200 (FIG. 2A) are sequenced using (long-read) sequencing methods to determine both the coupling of barcodes 114 and, optionally, some portion of the intervening template sequence. Because each barcoded bead interacts with <1 template on average, it is likely that that the intervening sequence fragments 210 associated with barcode i all derive from the same original template molecule, and likewise, via the coupling of i to j, and j to k, that any sequence fragments 210 associated with i, j, or k derives from the same template. Moreover, because i associates only to j, and j associates only to k, and because in this non-limiting example the barcodes are physically attached to a substrate that enables any given barcode to interact with only a local region of the long template molecule (as opposed to moving freely in a droplet reaction), a partial ordering of the fragments 210 can be inferred as shown in FIG. 2B. Specifically, the ordering is i - - - i<i - - - j<j - - - j<j - - - k<k - - - k. This ordering information can help to resolve certain types of genomic repeat structures, such as the Higher Order Repeats (HORs) present in mammalian centromeres.

Thus, in this method long fragments of DNA are contacted with multiple barcoded physical structures, which allow for the generation of long or paired-end reads that contain two barcodes 214 as shown in FIG. 2A. Accordingly, a polynucleotide of interest may be covered by multiple barcodes. In some embodiments, if the physical structure is a bead, then pairs of barcodes may identify “bead hopping” segments of the polynucleotide of interest as shown in FIG. 2B. The spatial information provided by the pairs of bead-tethered barcodes can reveal the long-range structure of the polynucleotide of interest. In some embodiments, using TELL-Seq™ beads provided 5-7 kbp linked barcoded fragments verified by secondary sequencing processes. Alternatively, in some embodiments, bead-primed multiple displacement amplification (MDA) may be employed, which is discussed below.

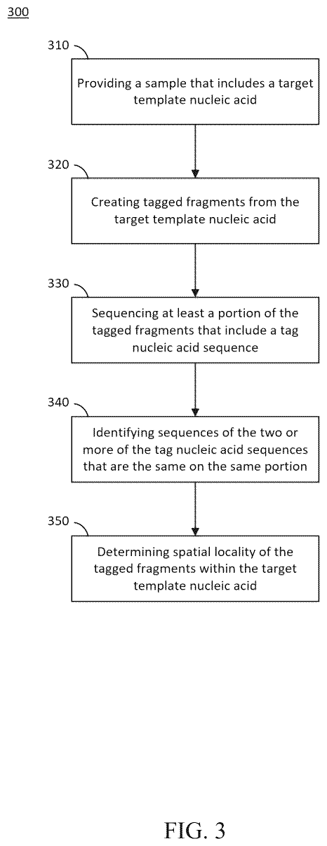

Some embodiments of the present disclosure relate to a method 300 of determining spatial locality of fragments derived from a same target template nucleic acid molecule is provided as shown in FIG. 3, the method including: a) providing a sample including a target template nucleic acid molecule, as shown in block 310, b) creating tagged fragments of the target template nucleic acid molecule using sets of tag nucleic acid molecules, as shown in block 320, wherein: i) each of the sets of tag nucleic acid molecules includes a tag portion, the tag portion including a tag nucleic acid sequence that is unique to that set; ii) the target template nucleic acid molecule is contacted with two or more different sets of the tag nucleic acid molecules; and iii) the tag nucleic acid molecules of each of the sets are spatially associated; wherein each of the tagged fragments include the tag nucleic acid sequence; c) sequencing at least a portion of the tagged fragments as shown in block 330, wherein said portion includes the tag nucleic acid sequence; d) identifying sequences of the tagged fragments that include two or more of the tag nucleic acid sequences that are the same, as shown in block 340, and e) determining the identified tagged fragments as likely originating from a same spatial locality of the target template nucleic acid molecule as shown in block 350.

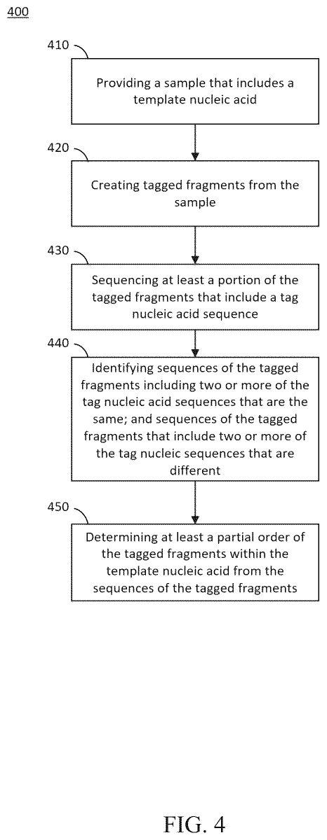

In some embodiments, a method 400 of determining at least a partial order of fragments derived from a same target template nucleic acid molecule is provided as shown in FIG. 4, the method including: a) providing a sample including a target template nucleic acid molecule as shown in block 410, b) creating tagged fragments of the target template nucleic acid molecule using sets of tag nucleic acid molecules, as shown in block 420, wherein: i) each of the sets of tag nucleic acid molecules includes a tag portion including a tag nucleic acid sequence that is unique to that set; il) the target template nucleic acid molecule is contacted with two or more different sets of tag nucleic acid molecules; and iii) the tag nucleic acid molecules of each of the sets are spatially associated; wherein each end of the tagged fragments includes the tag nucleic acid sequence; c) sequencing at least a portion of the tagged fragments, as shown in block 430, wherein said portion includes the tag nucleic acid sequence; d) identifying sequences of the tagged fragments that include two or more of the tag nucleic acid sequences that are same, and identifying sequences of the tagged fragments that include two or more of the tag nucleic acid sequences that are different as shown in block 440, and e) identifying sequences of the tagged fragments to determine the partial order of the tagged fragments with the target template nucleic acid molecule, as shown in block 450, wherein: a same first tag nucleic acid sequence at each end likely originated from a same first region of the target template nucleic acid molecule, a same second tag nucleic acid sequence at each end likely originated from a same second region of the target template nucleic acid molecule, and sequences of the tagged fragments which include the first tag nucleic acid sequence at one end and the second tag nucleic acid molecule at the other end originated from a region of the target template nucleic acid molecule intermediate to the first and second regions.

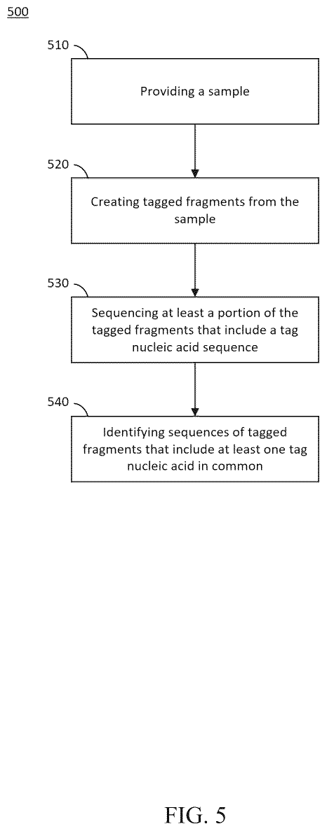

In some embodiments, a method of identifying fragments derived from a same target template nucleic acid molecule is provided in FIG. 5, the method including: a) providing a sample as shown in block 510, the sample including two or more target template nucleic acid molecules; and b) creating tagged fragments of the two or more target template nucleic acid molecules using sets of tag nucleic acid molecules as shown in block 520 Further, in step b) as shown in block 520, i) each of the sets of tag nucleic acid molecules includes a tag portion including a tag nucleic acid sequence that is unique to that set; ii) a modal number of different sets of tag nucleic acid molecules which contact each target template nucleic acid molecule is two or more, wherein each set of tag nucleic acid molecules includes a different tag nucleic acid sequence; and iii) a modal number of target template nucleic acid molecules which each set of tag nucleic acid molecules contacts is one, wherein each tagged fragment includes one or more tag nucleic acid sequences. The method further includes c) sequencing at least a portion of the tagged fragments, as shown in block 530, wherein said portion includes the tag nucleic acid sequence; and d) identifying sequences of the tagged fragments which have at least one tag nucleic acid sequence in common as shown in block 540 In some embodiments, the tagged fragments include a tag nucleic acid sequence at each end.

In some embodiments, step b) includes an amplification step, wherein the tag nucleic acid molecules are primers and include a target binding site capable of hybridising to at least one internal region of a target template nucleic acid molecule, and a tag portion, wherein the tag portion is 5′ to the target binding site. In some embodiments, the amplification step includes PCR amplification. In some embodiments, the amplification step includes isothermal amplification. In some embodiments, the amplification step includes multiple displacement amplification (MDA).

In some embodiments, at least one set of the tag nucleic acid molecules includes tag nucleic acid molecules having two or more different target binding sites. In some embodiments, the target binding sites include degenerate sequences. In some embodiments, the tag nucleic acid molecules are localised in a droplet. In some embodiments, two or more different sets of the tag nucleic acid molecules are located in each droplet.

In some embodiments, the tagged fragments include the tag nucleic acid sequence at each end similar to fragments shown in FIG. 2A. In some embodiments, step d) includes: linking 1) any sequences of the tagged fragments which include a same tag nucleic acid sequence at each end with 2) any sequences of the tagged fragments which include a different tag nucleic acid sequence at each end, wherein one of the different tag nucleic acid sequences is common with the same tag nucleic acid sequences; and determining the order of the tagged fragments within the target template nucleic acid molecule. In some embodiments, the sample further includes one or more additional target template nucleic acid molecules, wherein step d) further includes identifying sequences of the tagged fragments generated from the step c), the sequences of the tagged fragments including at least one of the tag nucleic acid sequence in common, and wherein the step e) further includes grouping the sequences of the tagged fragments that include at least one of the tag nucleic acid sequences in common to determine the spatial locality of the tagged fragments derived from the same target template nucleic acid molecule.

In some embodiments, the creating of the tagged fragments includes transposon-mediated fragmentation. In some embodiments, the tag nucleic acid molecules are immobilised on a solid support. In some embodiments, the sequencing step includes ligating the ends of the tagged fragments and sequencing the tag nucleic acid sequence in a region of the ligation junction, optionally wherein the sequencing includes sequencing at least 20, at least 30, at least 40, at least 50, at least 60, at least 70, at least 80, at least 90, or at least 100 nucleotides 5′ and/or 3′ of the ligation junction.

In some embodiments, each of the tag nucleic acid molecules includes a common adapter sequence 5′ to the tag portion, wherein each of the tagged fragments includes adapter sequences at 3′ and 5′ ends of the tagged fragment. In some embodiments, the adapter sequences can anneal to one another. In some embodiments, the method further includes a step of amplifying the tagged fragments using primers that are complementary to a portion of the adapter sequence at the 3′ end of each of the tagged fragments. In some embodiments, each of the tag nucleic acid molecules includes an adapter sequence 5′ to the tag portion and an adapter sequence 3′ to the tag portion, wherein the adapter sequence 5′ to the tag portion and the adapter sequence 3′ to the tag portion are the same sequence, and wherein each of the tagged fragments further includes, from each of the 5′ and 3′ ends thereof, a 5′ adapter sequence, the tag nucleic acid sequence, and a 3′ adapter sequence.

In some embodiments, after creating the tagged fragments, the method further includes: extending the 3′ end of any of the tagged fragments, for which the adapter sequence at the 3′ end of the tagged fragment has annealed to the adapter sequence at the 5′ end of the tagged fragment, using a 5′ tag nucleic acid sequence as an extension template to form a concatemeric sequence including the 5′ and a 3′ tag nucleic acid sequences at the 3′ end of the tagged fragment; and sequencing the concatemeric sequence. In some embodiments, the sequencing includes paired-end sequencing. In some embodiments, the sequencing further includes a step of bridge PCR. In some embodiments, the sequencing includes long read sequencing. In some embodiments, the sequencing includes nanopore sequencing. In some embodiments, the sequencing includes circular consensus sequencing. In some embodiments, the sequencing is synthetic long read sequencing. In some embodiments, the sequencing further includes determining the sequence of the at least one target nucleic acid molecule.

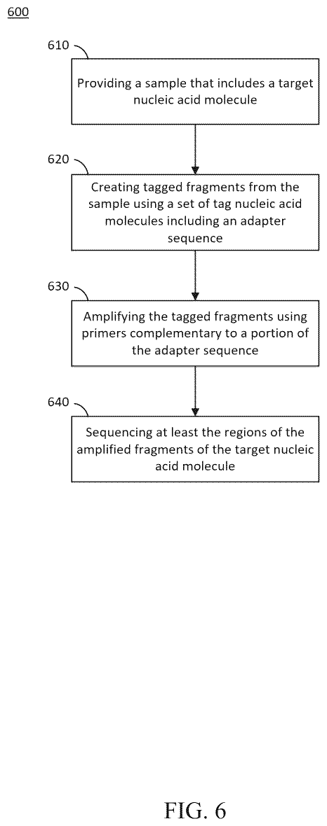

In some embodiments, a method for determining a sequence of at least one target nucleic acid molecule is provided as shown in FIG. 6, the method including: a) providing a sample including at least one target nucleic acid molecule as shown in block 610, b) creating tagged fragments of the target template nucleic acid molecule using a set of tag nucleic acid molecules, wherein the tag nucleic acid molecules include a single adapter sequence, as shown in block 620, wherein each of the fragments includes adapter sequences at 3′ and 5′ ends of the fragments; c) amplifying the fragments generated in step b), as shown in block 630, using primers that are complementary to a portion of adapter sequence at the 3′ end of each of the fragments; and d) sequencing at least regions of the amplified fragments of the target nucleic acid molecule generated in step c), as shown in block 640, wherein the tag nucleic acid molecules are conjugated to a solid support, wherein the fragments are created by tagmentation, and wherein the adapter sequences at the 3′ and 5′ ends of the fragments can anneal to one another. In some embodiments, the tag nucleic acid molecules are >1 kb (e.g., 1.5, 2, 2.5, 3, 3.5, 4, 4.5, 5 kb, or more)—the tag nucleic acid molecules conjugated on the solid support. In some embodiments, the tag nucleic acid molecules are >2 kb (e.g., 2.5, 3, 3.5, 4, 4.5, 5 kb, or more)—the tag nucleic acid molecules conjugated on the solid support.

In some embodiments, a method for determining a sequence of at least one target nucleic acid molecule is provided, the method including: a) providing a sample including at least one target nucleic acid molecule; b) creating tagged fragments of the at least one target nucleic acid molecule using sets of tag nucleic acid molecules, wherein: i) each of the sets of tag nucleic acid molecules includes: a tag portion including a tag nucleic acid sequence that is unique to that set; and a single adapter sequence, wherein the tag nucleic acid molecules are conjugated to a solid support; ii) the target template nucleic acid molecule is contacted with one or more different sets of tag nucleic acid molecules; and iii) the tag nucleic acid molecules of each of the sets are spatially associated; wherein the fragments are created by tagmentation, wherein each of the fragments includes adapter sequences at 3′ and 5′ ends of the fragments (the adapter sequences constructed so that can anneal to one another), and wherein each of the fragments derived from a same at least one target nucleic acid molecule include a same barcode sequence; c) amplifying the fragments using primers that are complementary to a portion of the adapter sequence at the 3′ end of each fragment; and d) sequencing at least regions of the amplified fragments of the target nucleic acid molecule generated in step c) to provide sequences of the fragments; and e) linking the sequences of the fragments which include the same barcode sequence, thereby to determine the sequence of the at least one target nucleic acid molecule. In some embodiments, the sequencing of step d) includes paired-end sequencing. In some embodiments, the sequencing of step d) includes a step of bridge PCR. In some embodiments, the sequencing of step d) includes long read sequencing.

In some embodiments, a method for determining a sequence of at least one target nucleic acid molecule is provided, the method including: a) providing a sample including at least one target nucleic acid molecule; b) creating tagged fragments of the at least one target nucleic acid molecule using sets of tag nucleic acid molecules, wherein: i) each of the sets of tag nucleic acid molecules includes a tag portion that includes: a tag nucleic acid sequence that is unique to that set; and a single adapter sequence; ii) the at least one target template nucleic acid molecule is contacted with one or more different sets of localised tag nucleic acid molecules; and iii) the tag nucleic acid molecules of each of the sets are spatially associated; wherein each of the fragments includes adapter sequences at 3′ and 5′ ends of the fragments that can anneal to one another, and wherein each of the fragments derived from a same set (or same sets) of the tagged nucleic acid molecules contacting with at least one target nucleic acid molecule includes the same barcode sequence(s). For example, tag nucleic acid molecules i (including a tag portion i) contacting the target nucleic acid molecule will lead to the creation of fragments with the same barcode sequence i. Likewise, tag nucleic acid molecules j (including a tag portion j) contacting the target nucleic acid molecule will lead to the creation of fragments with the same barcode sequence j. Additionally, if tag nucleic acid molecules i (including the tag portion i) and tag nucleic acid molecules j (including the tag portion j) contact the target nucleic acid molecule, then fragments with the same barcode sequences i and j may be created depending upon the spatial locality of tag nucleic acid molecules i and j contacting the target nucleic acid molecule.

In some embodiments, the method further includes c) amplifying the fragments generated in step ii) using primers that are complementary to a portion of the adapter sequence at the 3′ end of each fragment; and d) sequencing at least regions of the amplified fragments of the target nucleic acid molecule generated in step c) by long-read sequencing to provide long read sequences of the fragments; and e) linking the long read sequences of the fragments to determine the sequence of the at least one target nucleic acid molecule, wherein the tag nucleic acid molecules are conjugated to a solid support, wherein the fragments are created by tagmentation, and wherein the long read sequences of the fragments include a same barcode sequence. In some embodiments, the tag nucleic acid molecules are >1 kb (e.g., 1.5, 2, 2.5, 3, 3.5, 4, 4.5, 5 kb, or more)—the tag nucleic acid molecules conjugated on the solid support. In some embodiments, the tag nucleic acid molecules are >2 kb (e.g., 2.5, 3, 3.5, 4, 4.5, 5 kb, or more)—the tag nucleic acid molecules conjugated on the solid support. In some embodiments, the sequencing includes nanopore sequencing. In some embodiments, the sequencing includes circular consensus sequencing. In some embodiments, the sequencing includes synthetic long read sequencing.

In some embodiments, a method for fragmenting at least one target nucleic acid molecule is provided, the method including: a) providing a sample including at least one target nucleic acid molecule; b) creating tagged fragments of the target template nucleic acid molecule using a set of tag nucleic acid molecules, wherein the tag nucleic acid molecules include a single adapter sequence, wherein each of the fragments includes adapter sequences at 3′ and 5′ ends of the fragments which can anneal to one another; c) amplifying the fragments generated in step b) using primers that are complementary to a portion of adapter sequence at the 3′ end of each fragment; and d) collecting the amplified fragments generated in step c), wherein the tag nucleic acid molecules are conjugated to a solid support, and wherein the fragments are created by tagmentation.

In some embodiments, the method further includes sequencing the fragments. In some embodiments, the tag nucleic acid molecules further include an adapter sequence 5′ to the tag portion. In some embodiments, after the creating of the tagged fragments step, the fragments are amplified using primers capable of hybridising to the adapter sequences.

In some embodiments, the target template nucleic acid molecule(s) are longer than 10, 20, 30, 40, 50, 60, 70, 80, 980, 100, 200, 300, 400, 500, or 750 kb. In some embodiments, the target template nucleic acid molecule(s) are longer than 1, 2, 3, 4, 5, 10, 20, 50, or 100 M b or greater in length.

In some embodiments, the sample includes at least 2, 3, 4, 5, 10, 20, 50, 100, 200, 500, 1000, 2000, 5000, 10,000, 20,000, 50,000 or 100,000 target template nucleic acid molecules.

In some embodiments, the method further includes mapping sequences of the fragments to a reference genome. In some embodiments, the method includes creating an assembly graph from the sequences of the fragments.

### Examples

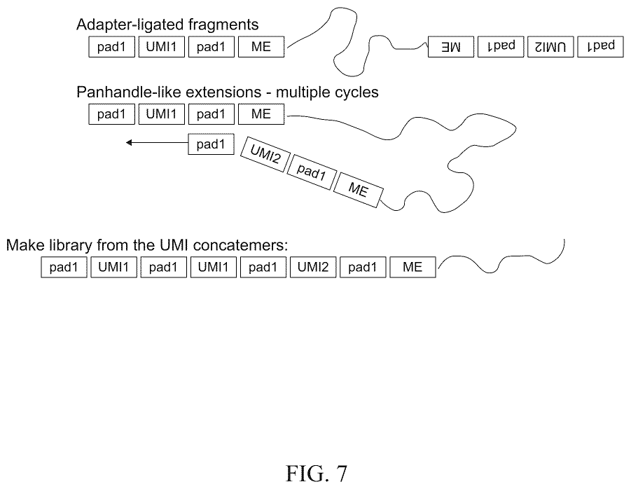

In a non-limiting example, FIG. 7 shows generation of tag pair concatemer sequences via template self-priming. Sequencing of the reaction products to identify pairings of unique identifiers (UMIs) can be accomplished using any of the following methods: (a) directly sequencing long fragments that contain the UMI on the fragment ends using a native long read or synthetic long read sequencing technology, (b) via a sample preparation method that involves circular ligation and selective sequencing of the ligation junctions, (c) via polymerase-mediated concatenation of the UMI on the ends of the fragments and selective sequencing of the concatemeric products as shown in FIG. 7. As shown, adapters with a structure similar to [pad1][UMI][pad1] are introduced to the 5′ and 3′ ends of a template. A thermal cycling reaction with a polymerase is then applied using conditions that favor templates folding back upon themselves, causing the 3′ end to anneal near the complementary sequences in the 5′ end. For fragments where the same UMI has been introduced to both the 3′ and 5′ ends, the most energetically favorable conformation will be for the entire adapter to anneal, preventing 3′ extension. For fragments with different UMIs, the pad1 sequences are more likely to anneal in a manner that permits 3′ polymerase extension to copy the 5′ UMI to the 3′ end of the template. Finally, a sequencing library can be constructed from the concatenated UMI tags, and optionally some portion of the template sequence.

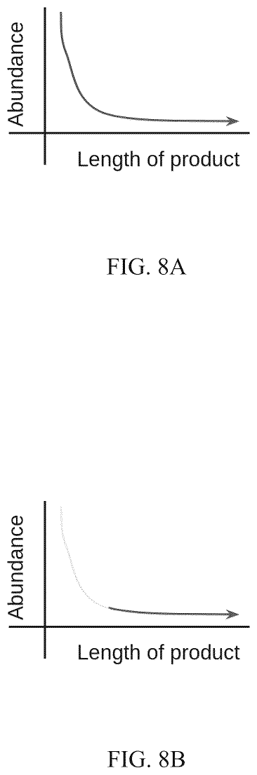

FIGS. 8A-B show line graphs comparing the abundance of products and length of products in another non-limiting example of long-linked reads via on-bead M DA. In this example, an isothermal MDA reaction is used to introduce identifiers into potentially overlapping copies of subsequences of a long template nucleic acid. MDA is known for producing extreme amplification bias, despite efforts to minimize the effect. When attempting to determine the sequence of a long template molecule by tagging, amplifying and sequencing portions of that template, it is highly desirable for the amplification process to produce uniform amplification throughout the template. Size selection is proposed as a mechanism to increase the uniformity of amplification. In FIG. 8A, regions of the sequence go through many rounds of MDA will tend to form short extension products because, on average, each round of extension will yield a product that is half the length of that produced by the previous round. Size selection shown in FIG. 8B to reduce M DA bias can be accomplished in a number of ways: 1) via suppression PCR, which strong favors amplification of longer products, thereby eliminating bias created by MDA that is present in short fragments; 2) via bead size selection using solid-phase reversible immobilization (SPRI) or Circulomics beads; or 3) via agarose gel separation.

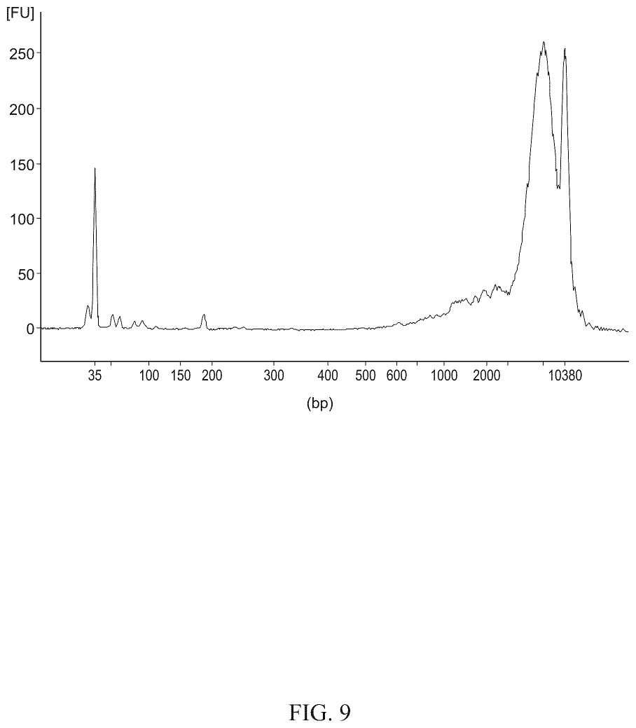

In another non-limiting example, when using a TELL-Seq™ bead workflow, short (˜200-1000 bp) single-barcoded linked read libraries are generated for sequencing. This workflow includes a step of initial tagmentation (Mu transposase) and ligation to barcoded bead oligos. A second tagmentation step further fragments the DNA and introduces a second priming site for library amplification. Modifying the TELL-Seq™ bead workflow for dual barcoded linked long templates omits the second tagmentation step. This modification allows for a custom PCR that uses a single primer to amplify long templates directly from TELL-Seq™ beads. Moreover, the modified TELL-Seq™ bead workflow preserves the bead barcode sequence that is introduced at each end of the template. FIG. 9 is illustrative where a small number of human gDNA template molecules (<100 k) in the size range 20-100 kbp reacted with barcoded tagmentation beads. The reaction products were subsequently amplified using a single primer to make many copies of each dual-tagged template. The modal fragment size of these dual tagged templates was about 7 kbp, as shown in a dominant product peak centered at ˜7 kbp in FIG. 9 from a sample that was run on an Agilent Bioanalyzer using a High Sensitivity DNA Kit. Thereafter, the ends of the dual tagged templates were sequenced with paired-end sequencing on an Illumina MiSeq™ instrument, including a portion of the template as well as the bead barcode introduced by the on-bead tagmentation. The reads were mapped to the human reference genome hg38. 4,008,650 mapped reads were properly paired (the paired-end reads mapping within the expected insert size distance, roughly 200-1000 nt apart). Nearby read pairs that map with opposite orientations and with mapping positions that are within the expected distance (3 to 10 kbp) are taken to be ends of the same long fragment, in particular when there are no other reads mapping to the intervening region.

In a non-limiting example, it was found that 32,949 reads from ends of tagged fragments faced each other and were between 3 kb and 10 kb apart, which suggests at least 32,949 long barcoded fragments were generated from the original set of long template nucleic acids. In the paired-end reads from the ends of a 7.8 kbp long bead-barcoded fragments, each 75 nt read data may be visualized. The design of the bead tagmentation adapter causes read 2 in a read pair to derive from the end of a long tagged fragment, while read 1 in the read pair starts at some arbitrary position a few hundred nucleotides into the tagged fragment. The collection of read 2's from the same tagged fragment end is referred to as an “endwall” because the reads all start at exactly the same position. This structure provides information about the orientation of a read pair with respect to the tagged fragment that it was derived from. In this example, the reads in blue come from one end of the 7.8 kbp tagged fragment while the reads in red come from the other end, with the blue and red read 2's having opposite mapping orientations. All reads contain the same bead barcode sequence.

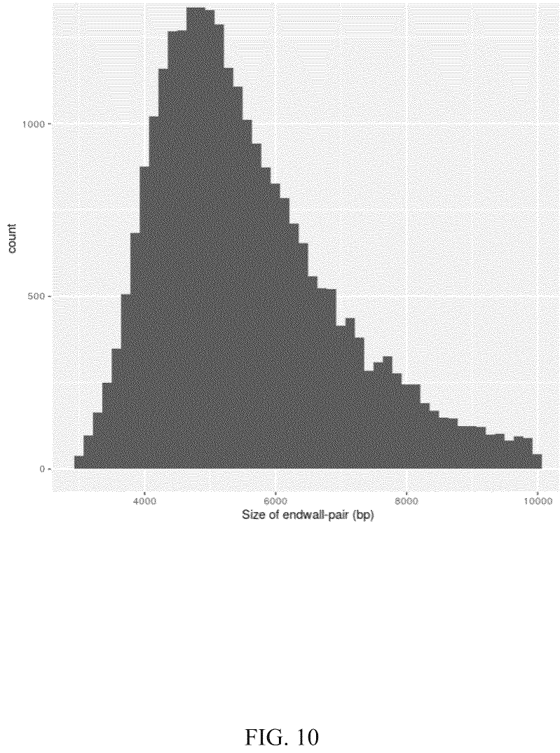

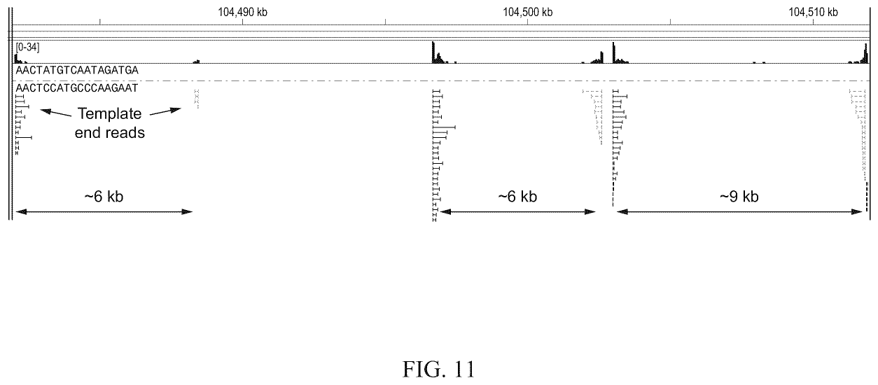

Nearby endwall pairs that contain mapped reads in the intervening region between the endwall pairs were filtered out as these could not be unambiguously assigned as ends of the same tagged fragment, which left 27,085 endwall pairs. The median distance between these endwall pairs was 5282 nt and their distribution of lengths is shown in FIG. 10. Furthermore, FIG. 11 shows an example of three tagged fragments that were likely derived from a common starting DNA molecule via tagmentation by a single barcoded bead. All three fragments shown in FIG. 11 contain the same barcode sequence on both ends, and are in close proximity within the genome. The results from FIG. 11 confirm template linkage and bead hopping. End libraries were generated via Nextera tagmentations of long tagged fragments and selective amplification of end fragments to preserve the TELL-Seq™ bead barcodes. The data was filtered to identify end pairs that were likely derived from the same long tagged fragment. The median fragment size was consistent with lab observations. FIG. 11 illustrates an example of multiple nearby fragments sharing the same bead barcode. A bout 10% of apparently genuine end pairs had different bead barcodes, which is indicative of bead hopping. Similar results were confirmed via nanopore sequencing of long fragments.

The bead barcodes that were associated with simple endwall pairs were examined and, of these, 24,675 had the same barcode in both endwalls, suggesting that both ends of the fragment were tagmented by the same barcoded bead. The remaining 2,410 fragments had different barcodes, suggesting that the fragment spanned two different tagmentation beads. A simulation was performed to compute the number of barcoded fragment ends with different bead barcodes that would be expected to appear associated with each other by chance. The simulation was compared against a model whereby fragments were uniformly distributed throughout the genome and there was a 50% chance that any individual fragment end would fail to be identified via the sample preparation and sequencing process. If it is assumed that there are 30,000 fragments uniformly distributed through the 3 Gbp human genome, and 50% of fragment ends fail to be identified, then it would be expected to find about 75 ends from different fragments that would be nearby, correctly oriented, and without an intervening endwall. If the count of fragments in the simulation was increased to 60,000, then it is expected to find about 285 endwall pairs that are due to false pairing from overlapping fragments. In both cases, the observed number of endwall pairs in the real data that associated different barcodes (2,410) greatly exceeded the number that would be expected to occur by chance. Therefore, the long read sequencing of these products, or another sequencing approach that enables direct coupling of the fragment ends (as described above), enables the path of a single molecule to be traced as it interacts with multiple barcoded beads.

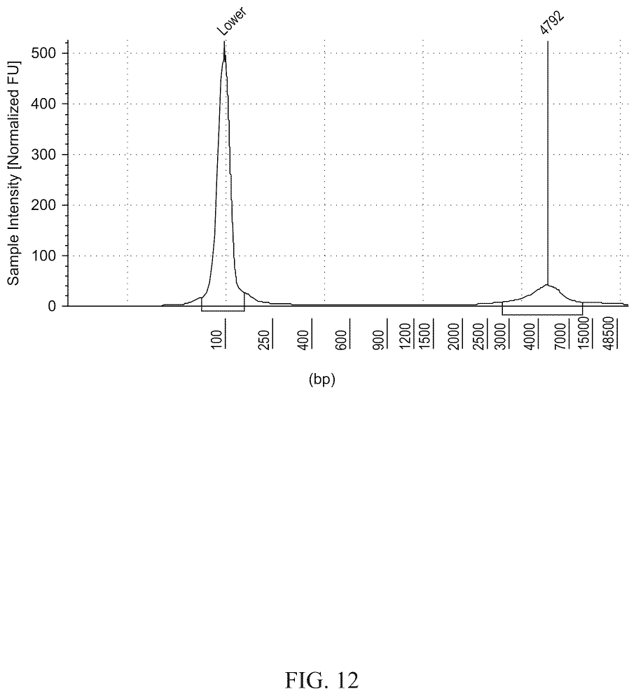

In another non-limiting example, results from a bead-primed multiple displacement amplification (MDA) are shown in FIG. 12. In this example, long-linked reads on-bead MDA were used to generate barcoded bead preparations using a handful of pre-via synthesized barcodes (which may be referred to as UMIs). The results came from random priming and MDA from bead-bound, barcoded oligonucleotides (no tagmentation). As shown, this MDA-based example supports overlapping linked templates and longer templates. So far, MDA products have been generated ˜5 kbp in size using biotinylated and adapter-tailed random oligonucleotides attached to streptavidin beads (no barcodes) as shown. End libraries of MDA templates have been prepared and sequenced, thereby confirming successful incorporation of sequencing adapters during the on-bead M DA step.

### Definitions

The section headings used herein are for organizational purposes only and are not to be construed as limiting the subject matter described.

Unless defined otherwise, all technical and scientific terms used herein have the same meaning as is commonly understood by one of ordinary skill in the art. The use of the term “including” as well as other forms, such as “include”, “includes,” and “included,” is not limiting. The use of the term “having” as well as other forms, such as “have”, “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

The term “Sample” as used herein is typically derived from a biological fluid, cell, tissue, organ, or organism, comprising a nucleic acid or a mixture of nucleic acids comprising at least one nucleic acid sequence that is to be sequenced. The sample may be used directly as obtained from the biological source or following a pretreatment to modify the character of the sample. For example, such pretreatment may include preparing plasma from blood, diluting viscous fluids and so forth. Methods of pretreatment may also involve, but are not limited to, filtration, precipitation, dilution, distillation, mixing, centrifugation, freezing, lyophilization, concentration, amplification, nucleic acid fragmentation, inactivation of interfering components, the addition of reagents, lysing, etc. If such methods of pretreatment are employed with respect to the sample, such pretreatment methods are typically such that the nucleic acid(s) of interest remain in the test sample, sometimes at a concentration proportional to that in an untreated test sample (e.g., namely, a sample that is not subjected to any such pretreatment method(s)).

The terms “polynucleotide,” “oligonucleotide,” “nucleic acid” and “nucleic acid molecules” are used interchangeably herein and refer to a covalently linked sequence of nucleotides of any length (i.e., ribonucleotides for RNA, deoxyribonucleotides for DNA, analogs thereof, or mixtures thereof) in which the 3′ position of the pentose of one nucleotide is joined by a phosphodiester group to the 5′ position of the pentose of the next. The terms should be understood to include, as equivalents, analogs of either DNA, RNA, cDNA, or antibody-oligo conjugates made from nucleotide analogs and to be applicable to single stranded (such as sense or antisense) and double stranded polynucleotides. The term as used herein also encompasses cDNA, that is complementary or copy DNA produced from a RNA template, for example by the action of reverse transcriptase. This term refers only to the primary structure of the molecule. Thus, the term includes, without limitation, triple-, double- and single-stranded deoxyribonucleic acid (“DNA”), as well as triple-, double- and single-stranded ribonucleic acid (“RNA”). The nucleotides include sequences of any form of nucleic acid.

The term “target nucleic acid” as used herein is intended as a semantic identifier for the nucleic acid in the context of a method or composition set forth herein and does not necessarily limit the structure or function of the nucleic acid beyond what is otherwise explicitly indicated. A target nucleic acid may be essentially any nucleic acid of known or unknown sequence. It may be, for example, a fragment of genomic DNA (e.g., chromosomal DNA), extra-chromosomal DNA such as a plasmid, circulating DNA or circulating RNA, nucleic acids from a cell or cells, cell-free DNA, RNA (e.g., mRNA), or cDNA. Sequencing may result in determination of the sequence of the whole, or a part of the target molecule. The targets can be derived from a primary nucleic acid sample, such as a nucleus. In one embodiment, the targets can be processed into templates suitable for amplification by the placement of universal sequences at the end or ends of each target fragment. The targets can also be obtained from a primary RNA sample by reverse transcription into cDNA. In one embodiment, target is used in reference to a subset of DNA or RNA in the cell. Targeted sequencing uses selection and isolation of genes of interest, typically by either PCR amplification (e.g. region-specific primers) or hybridization-based capture method or antibodies. Targeted enrichment can occur at various stages of the method. For instance, a targeted RNA representation can be obtained using target specific; primers in the reverse transcription step or hybridization-based enrichment of a subset out of a more complex library. An example is exome sequencing or the L 1000 assay (Subramanian et al., 2017, Cell, 171; 1437-1452). Targeted sequencing can include any of the enrichment processes including target enrichment, hybridization capture-based target enrichment, enrichment via molecular inversion probes (MIP), primer extension target enrichment (PETE), amplicon-based enrichment, CRISPR/Cas9-based targeted enrichment, in silico enrichment, or the like. A target nucleic acid having a universal sequence one or both ends can be referred to as a modified target nucleic acid. Reference to a nucleic acid such as a target nucleic acid includes both single stranded and double stranded nucleic acids unless indicated otherwise. For instance, symmetric and asymmetric target nucleic acids can be double-stranded, single stranded, or partly double and single stranded at some point in the method of the present disclosure.

The term “adapter” and its derivatives as used herein refers generally to any linear oligonucleotide which can be attached to a target nucleic acid. A n adapter can be single-stranded or double-stranded DNA, or can include both double stranded and single stranded regions. An adapter can include a universal sequence that is substantially identical, or substantially complementary, to at least a portion of a primer, for example a universal primer. In some embodiments, the adapter is substantially non-complementary to the 3′ end or the 5′ end of any target sequence present in the sample. In some embodiments, suitable adapter lengths are in the range of about 6-100 nucleotides, about 12-60 nucleotides, or about 15-50 nucleotides in length. For instance, the terms “adaptor” and “adapter” are used interchangeably.

The term “primer” and its derivatives as used herein refer generally to any nucleic acid that can hybridize to a target sequence of interest. Typically, the primer functions as a substrate onto which nucleotides can be polymerized by a polymerase or to which a polynucleotide can be ligated; in some embodiments, however, the primer can become incorporated into the synthesized nucleic acid strand and provide a site to which another primer can hybridize to prime synthesis of a new strand that is complementary to the synthesized nucleic acid molecule. The primer can include any combination of nucleotides or analogs thereof.

The term “barcode,” “unique molecular identifier” (or “UMI”), or a “tag” as used interchangeably herein refers to a unique nucleic acid tag, either random, non-random, or semi-random, that can be used to identify a sample or source of the nucleic acid material, or a compartment in which a target nucleic acid was present. The barcode can be present in solution or on a solid-support, or attached to or associated with a solid-support and released in solution or compartment. When nucleic acid samples are derived from multiple sources, the nucleic acids in each nucleic acid sample can be tagged with different nucleic acid tags such that the source of the sample can be identified. Any suitable barcode or set of barcodes can be used, as exemplified by the disclosures of U.S. Pat. No. 8,053,192, PCT Publication No. WO 05/068656, and U.S. Pat. Publication No. 2013/0274117.

The term “amplicon” as used herein, when used in reference to a nucleic acid, means the product of copying the nucleic acid, wherein the product has a nucleotide sequence that is the same as or complementary to at least a portion of the nucleotide sequence of the nucleic acid. An amplicon can be produced by any of a variety of amplification methods that use the nucleic acid, or an amplicon thereof, as a template including, for example, polymerase extension, polymerase chain reaction (PCR), rolling circle amplification (RCA), ligation extension, ligation chain reaction, or multiple displacement amplification (MDA). An amplicon can be a nucleic acid molecule having a single copy of a particular nucleotide sequence (e.g., a PCR product) or multiple copies of the nucleotide sequence (e.g., a concatameric product of RCA). A first amplicon of a target nucleic acid is typically a complimentary copy. Subsequent amplicons are copies that are created, after generation of the first amplicon, from the target nucleic acid or from the first amplicon. A subsequent amplicon can have a sequence that is substantially complementary to the target nucleic acid or substantially identical to the target nucleic acid.

The term “tagmentation” refers to the modification of DNA by a transposome complex comprising transposase enzyme complexed with adaptors comprising transposon end sequence. Tagmentation results in the simultaneous fragmentation of the DNA and ligation of the adaptors to the 5′ ends of both strands of duplex fragments. Following a purification step to remove the transposase enzyme, additional sequences can be added to the ends of the adapted fragments, for example by PCR, ligation, or any other suitable methodology known to those having skill in the art.

The terms “amplify”, “amplifying” or “amplification reaction” and their derivatives, as used herein, refer generally to any action or process whereby at least a portion of a nucleic acid molecule is replicated or copied into at least one additional nucleic acid molecule. The additional nucleic acid molecule optionally includes sequence that is substantially identical or substantially complementary to at least some portion of the template nucleic acid molecule. The template nucleic acid molecule can be single-stranded or double-stranded and the additional nucleic acid molecule can independently be single-stranded or double-stranded. Amplification optionally includes linear or exponential replication of a nucleic acid molecule. In some embodiments, such amplification can be performed using isothermal conditions; in other embodiments, such amplification can include thermocycling. In some embodiments, the amplification is a multiplex amplification that includes the simultaneous amplification of a plurality of target sequences in a single amplification reaction. In some embodiments, “amplification” includes amplification of at least some portion of DNA and RNA based nucleic acids alone, or in combination.

As used herein, the term “polymerase chain reaction” (“PCR”) refers to the method of Mullis U.S. Pat. Nos. 4,683,195 and 4,683,202, which describe a method for increasing the concentration of a segment of a polynucleotide of interest in a mixture of genomic DNA without cloning or purification. This process for amplifying the polynucleotide of interest consists of introducing a large excess of two oligonucleotide primers to the DNA mixture containing the desired polynucleotide of interest, followed by a series of thermal cycling in the presence of a DNA polymerase. The two primers are complementary to their respective strands of the double stranded polynucleotide of interest. The mixture is denatured at a higher temperature first and the primers are then annealed to complementary sequences within the polynucleotide of interest molecule. Following annealing, the primers are extended with a polymerase to form a new pair of complementary strands. The steps of denaturation, primer annealing and polymerase extension can be repeated many times (referred to as thermocycling) to obtain a high concentration of an amplified segment of the desired polynucleotide of interest. The length of the amplified segment of the desired polynucleotide of interest (amplicon) is determined by the relative positions of the primers with respect to each other, and therefore, this length is a controllable parameter. By virtue of repeating the process, the method is referred to as PCR. Because the desired amplified segments of the polynucleotide of interest become the predominant nucleic acid sequences (in terms of concentration) in the mixture, they are said to be “PCR amplified”. In a modification to the method discussed above, the target nucleic acid molecules can be PCR amplified using a plurality of different primer pairs, in some cases, one or more primer pairs per target nucleic acid molecule of interest, thereby forming a multiplex PCR reaction.

The term “multiple displacement amplification” (MDA) as used herein refers to an isothermal strand-displacing replication by multiple primers. In some embodiments, the primers include random hexamers, which is an oligonucleotide of a random sequence of six nucleotides.

### Additional Notes

The tagging reaction can be mediated by any method, including tagmentation, ligation, or polymerase extension of an oligo containing a tag. It can occur via uniquely barcoded bead-or surface-bound transposomes in solution, bead-or surface-bound oligos in solution, or in reaction droplets in an emulsion or microfluidic device. The polymerase extension can be carried out using thermal cycling with any polymerase or in an isothermal reaction using a strand displacing polymerase, and can be used either in a linear extension or a rolling circle replication modality or via a loop-mediated isothermal amplification modality. Amplification bias can be reduced using size selection to remove short fragments (e.g., <1 kbp, <5 kbp, or a gradient up to 10 kbp), using any size selection method including (but not limited to) bead size selection, gel size selection, or single primer PCR amplification under conditions that preferentially amplify long fragments.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

It is to be understood that the ranges provided herein include the stated range and any value or sub-range within the stated range, as if such value or sub-range were explicitly recited. For example, a range from about 2 kbp to about 20 kbp should be interpreted to include not only the explicitly recited limits of from about 2 kbp to about 20 kbp, but also to include individual values, such as about 3.5 kbp, about 8 kbp, about 18.2 kbp, etc., and sub-ranges, such as from about 5 kbp to about 10 kbp, etc. Furthermore, when “about” and/or “substantially” are/is utilized to describe a value, this is meant to encompass minor variations (up to +/−10%) from the stated value.

While several examples have been described in detail, it is to be understood that the disclosed examples may be modified. Therefore, the foregoing description is to be considered non-limiting.

While certain examples have been described, these examples have been presented by way of example only, and are not intended to limit the scope of the disclosure. Indeed, the novel methods described herein may be embodied in a variety of other forms. Furthermore, various omissions, substitutions and changes in the methods described herein may be made without departing from the spirit of the disclosure. The accompanying claims and their equivalents are intended to cover such forms or modifications as would fall within the scope and spirit of the disclosure.

Features, materials, characteristics, or groups described in conjunction with a particular aspect, or example are to be understood to be applicable to any other aspect or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing examples. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations, one or more features from a claimed combination can, in some cases, be excised from the combination, and the combination may be claimed as a sub-combination or variation of a sub-combination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some examples, the actual steps taken in the processes illustrated and/or disclosed may differ from those shown in the figures. Depending on the example, certain of the steps described above may be removed or others may be added. Furthermore, the features and attributes of the specific examples disclosed above may be combined in different ways to form additional examples, all of which fall within the scope of the present disclosure.

For purposes of this disclosure, certain aspects, advantages, and novel features are described herein. Not necessarily all such advantages may be achieved in accordance with any particular example. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain examples include, while other examples do not include, certain features, elements, and/or steps. Thus, such conditional language is not generally intended to imply that features, elements, and/or steps are in any way required for one or more examples or that one or more examples necessarily include logic for deciding, with or without user input or prompting, whether these features, elements, and/or steps are included or are to be performed in any particular example.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain examples require the presence of at least one of X, at least one of Y, and at least one of Z.

Language of degree used herein, such as the terms “approximately,” “about,” “generally,” and “substantially” represent a value, amount, or characteristic close to the stated value, amount, or characteristic that still performs a desired function or achieves a desired result.

The scope of the present disclosure is not intended to be limited by the specific disclosures of preferred examples in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A method of determining at least a partial order of fragments derived from a same target template nucleic acid molecule, said method comprising:
a) providing a sample comprising a target template nucleic acid molecule;
b) creating tagged fragments of the target template nucleic acid molecule using sets of tag nucleic acid molecules, wherein:
i) each of the sets of tag nucleic acid molecules comprises a tag portion comprising a tag nucleic acid sequence that is substantially unique to that set;
ii) the target template nucleic acid molecule is contacted with two or more different sets of tag nucleic acid molecules; and
iii) the tag nucleic acid molecules of each of the sets are spatially associated;
wherein each end of the tagged fragments includes the tag nucleic acid sequence;
c) sequencing at least a portion of the tagged fragments, wherein said portion includes the tag nucleic acid sequence;
d) identifying sequences of the tagged fragments that include two or more of the tag nucleic acid sequences that are same, and identifying sequences of the tagged fragments that include two or more of the tag nucleic acid sequences that are different; and
e) identifying sequences of the tagged fragments to determine the partial order of the tagged fragments to the target template nucleic acid molecule, wherein:
a same first tag nucleic acid sequence at each end of the tagged fragment likely originated from a same first region of the target template nucleic acid molecule,
a same second tag nucleic acid sequence at each of the ends likely originated from a same second region of the target template nucleic acid molecule, and
sequences comprising the first tag nucleic acid sequence at one of the ends and the second tag nucleic acid molecule at the other end as likely originating from a region of the target template nucleic acid molecule that is intermediate to the first and second regions.

2. The method of claim 1, wherein the tagged fragments comprise a tag nucleic acid sequence at each end.

3. The method of claim 1, wherein step b) comprises an amplification step, wherein the tag nucleic acid molecules are primers and comprise a target binding site capable of hybridising to at least one internal region of a target template nucleic acid molecule, and a tag portion, wherein the tag portion is 5′ to the target binding site.

4. The method of claim 3, wherein at least one set of the tag nucleic acid molecules comprises tag nucleic acid molecules having two or more different target binding sites.

5. The method of claim 4, wherein the target binding sites include degenerate sequences.

6. The method of claim 1, wherein the creating of the tagged fragments comprises transposon-mediated fragmentation.

7. The method of claim 1, wherein the tag nucleic acid molecules are immobilised on a solid support.

8. The method of claim 1, wherein the sequencing step comprises ligating the ends of the tagged fragments and sequencing the tag nucleic acid sequence in a region of the ligation junction, optionally wherein the sequencing comprises sequencing at least 20, at least 30, at least 40, at least 50, at least 60, at least 70, at least 80, at least 90, or at least 100 nucleotides 5′ and/or 3′ of the ligation junction.

9. The method of claim 1, wherein each of the tag nucleic acid molecules comprises a common adapter sequence 5′ to the tag portion, wherein each of the tagged fragments comprises adapter sequences at 3′ and 5′ ends of the tagged fragment, wherein the adapter sequences can anneal to one another.

10. The method of claim 9, wherein the method further comprises a step of amplifying the tagged fragments using primers that are complementary to a portion of the adapter sequence at the 3′ end of each of the tagged fragments.

11. The method of claim 10, wherein each of the tag nucleic acid molecules comprises an adapter sequence 5′ to the tag portion and an adapter sequence 3′ to the tag portion,
wherein the adapter sequence 5′ to the tag portion and the adapter sequence 3′ to the tag portion are the same sequence, and
wherein each of the tagged fragments further includes, from each the 5′ and 3′ ends thereof, a 5′ adapter sequence, the tag nucleic acid sequence, and a 3′ adapter sequence.

12. The method of claim 11, wherein after creating the tagged fragments, the method further comprises:
extending the 3′ end of any of the tagged fragments, for which the adapter sequence at the 3′ end of the tagged fragment has annealed to the adapter sequence at the 5′ end of the tagged fragment, using a 5′ tag nucleic acid sequence as an extension template to form a concatemeric sequence comprising the 5′ and a 3′ tag nucleic acid sequences at the 3′ end of the tagged fragment; and
sequencing the concatemeric sequence.

13. The method of claim 1, further comprising determining the sequence of the at least one target nucleic acid molecule.

14. The method of claim 1, wherein the tag nucleic acid molecules further comprise an adapter sequence 5′ to the tag portion.

15. The method of claim 14, wherein, after the creating of the tagged fragments step, the fragments are amplified using primers capable of hybridising to the adapter sequences.

16. The method of claim 1, wherein any of the target template nucleic acid molecule are longer than 10 kb, longer than 20 kb, longer than 30 kb, longer than 40 kb, longer than 50 kb, longer than 60 kb, longer than 70 kb, longer than 80 kb, longer than 90 kb, longer than 100 kb, longer than 200 kb, longer than 300 kb, longer than 400 kb, longer than 500 kb, longer than 750 kb, longer than 1 Mb, longer than 2 Mb, longer than 3 Mb, longer than 4 Mb, longer than 5 Mb, longer than 10 Mb, longer than 20 Mb, longer than 50 Mb, or longer than 100 Mb in length.

17. The method of claim 1, wherein the sample comprises at least 2, at least 3, at least 4, at least 5, at least 10, at least 20, at least 50, at least 100, at least 200, at least 500, at least 1000, at least 2000, at least 5000, at least 10,000 at least 20,000 at least 50,000, or at least 100,000 target template nucleic acid molecules.

18. The method of 1, further comprising mapping sequences of the fragments to a reference genome.

19. The method of claim 1, further comprising creating an assembly graph from the sequences of the fragments.
